(19) United States
(12) Patent Application Publication
(10) Pub. No.: US 2016/0262718 A1
(43) Pub. Date: Sep. 15, 2016

(54) DISLOCATION SENSOR

(71) Applicant: BIOTRONIK SE & CO. KG, Berlin (DE)

(72) Inventors: Thomas DOERR, Berlin (DE); Uwe STUERMER, Berlin (DE)

(73) Assignee: BIOTRONIK SE & CO. KG, Berlin (DE)

(21) Appl. No.: 15/043,735

(22) Filed: Feb. 15, 2016

Related U.S. Application Data
(60) Provisional application No. 62/132,514, filed on Mar. 13, 2015.

(51) Int. Cl. A61B 8/08

(52) U.S. Cl. CPC A61B 8/0833; A61B 8/0883

(57) ABSTRACT

Embodiments include an active implantable medical therapy and/or monitoring system that includes at least one implant. The at least one implant includes at least one ultrasonic transducer directly or indirectly connected to a control and evaluation unit in order to emit and receive ultrasonic signals. The control and evaluation unit prompts an emission of ultrasonic signals by the at least one ultrasonic transducer cyclically or in a triggered manner and evaluates received ultrasonic signals such that the control and evaluation unit identifies an actual position or change in position of one or more of the at least one implant and an electrode line.

This application claims the benefit of U.S. Provisional Patent Application 62/132,514 filed on 13 Mar. 2015, the specification of which is hereby incorporated herein by reference.

### BACKGROUND OF THE INVENTION

1. Field of the Invention

Embodiments of the invention generally relate to an active implantable medical therapy and/or monitoring system that includes at least one implant.

2. Description of the Related Art

Typically, active implantable medical therapy and/or monitoring systems include implantable heart therapy and/or heart monitoring devices, for example cardiac pacemakers or cardioverters/defibrillators. Generally, cardiac pacemakers are often therapy systems that have an implantable pulse generator (the actual cardiac pacemaker) and electrode lines that are connected thereto and that have electrode poles.

A dedicated electrode line is typically provided for each respective heart chamber to be stimulated (for example right or left ventricle or right or left atrium).

Generally, a cardiac pacemaker may deliver an electric stimulation pulse to the muscle tissue of a respective heart chamber via one or more stimulation electrode poles of a respective electrode line in order to thus cause a stimulated contraction of the heart chamber, provided the stimulation pulse has a sufficient intensity and the heart muscle tissue (myocardium) is not in a refractory phase at that precise moment.

In addition, typically, the electrode lines with their electrode poles are also used to detect contractions of a respective heart chamber. Generally, the detection of natural events is used in demand pacemakers for example for the suppression (inhibition) of the delivery of stimulation pulses to a corresponding heart chamber, if the natural event is detected in a time window prior to the planned delivery of a stimulation pulse to this heart chamber.

In an electrocardiogram, typically, action potentials accompanying a contraction of the ventricle and reflecting a depolarization of the heart muscle cells are to be identified as a Q-wave, whereas the repolarization of the heart muscle cells accompanying the relaxation of the myocardium is reflected as a T-wave.

In healthy individuals, generally, the respective heart rhythm is determined by the sinus node, which is controlled by the autonomous nervous system. Typically, the sinus node excites the right atrium of a human heart by stimulus conduction and also excites the (right) ventricle of the heart via the AV node. Generally, a natural heart rhythm starting from the sinus node is therefore also referred to as the sinus rhythm and leads to natural contractions of the respective heart chamber, which may be detected as natural (intrinsic) events.

Typically, such natural (intrinsic) events are detected by recording the electric potentials of the myocardium of the respective heart chamber with the aid of sensing electrodes, which are part of a corresponding electrode line. Generally, the sensing electrode poles may be simultaneously the stimulation electrode poles and may be used alternately as a stimulation electrode pole and as a sensing electrode pole. Typically, a sensing electrode pole pair, which is formed by two adjacent electrode poles, specifically a point electrode (tip electrode) and a ring electrode, the point electrode also serving as a stimulation electrode pole, is typically provided for sensing, such as the sensing of intrinsic events. Generally, a bipolar recording of an intracardial electrocardiogram (IEGM) may be provided. Typically, intrinsic events and the stimulation in the ventricle are sensed with the aid of a ventricular electrode line, and the stimulation and the sensing of intrinsic events in the atrium (in the right atrium) are implemented with an atrial electrode line, wherein electrode lines are connected separately to the respective heart stimulator. In addition, generally, a left-ventricular electrode line may also be provided, which typically protrudes via the coronary sinus and a lateral vein branching off therefrom into the vicinity of the left ventricle, where it may have a small-area stimulation and/or sensing electrode.

Generally, regarding references used herein, it should be noted that the terms stimulation electrode or sensing electrode within the scope of the invention may refer to a respective electrode pole on an electrode line, for example the part of an electrode line via which stimulation pulses are delivered or electric potentials are received. It should also be noted that, generally, an electrode line used for stimulation may be referred to as a “stimulation electrode”.

Typically, the sensing electrode poles are connected during operation of the heart stimulator to corresponding sensing units, which may evaluate a respective electrocardiogram recorded via a sensing electrode pole (or a sensing electrode pole pair) and in particular may detect intrinsic atrial or ventricular events, such as natural atrial or ventricular contractions. Generally, this is achieved by way of example using a threshold value comparison, for example an intrinsic event is detected when a respective intracardial electrocardiogram exceeds a suitably predefined threshold value.

Typically, as a result of the dislocation (“slipping” or “shifting”) of an electrode line together with its sensing electrode poles, amplitudes and/or the form of the signals recorded via one or more sensing electrode poles may change without this being caused physiologically. Generally, this poses a problem with regard to a reliable evaluation of detected signals, and it is therefore desired to identify a dislocation of an electrode line and/or electrode poles thereof, especially to derive a series of relevant therapy parameters from detected signals.

Typically, with regard to a possible dislocation of sensing electrode poles influencing the sensing of events, several solution approaches have been used in order to identify such a dislocation. Generally, no solution approaches for identifying a dislocation of a stimulation electrode are based on the evaluation of the sensing amplitude, the electrode impedances and the stimulation thresholds.

For example, U.S. Pat. No. 7,664,550 to Eick et al., entitled “Method and Apparatus for Detecting Left Ventricular Lead Displacement Based Upon EGM Change”, describes a method for identifying the dislocation of a left-ventricular electrode line with electrode poles thereof on the basis of a modified signal amplitude, morphology or a modified time interval from the atrial signal.

Typically, previous approaches for identifying electrode position errors utilize the measurement and evaluation of the following parameters: signal amplitudes of biosignals, impedances, stimulus thresholds, and signal form analyses (also comparison in a number of channels).

### BRIEF SUMMARY OF THE INVENTION

One or more embodiments of the invention may timely and reliably identify an inadmissible change in position of one or more of an electronic implant and an implanted electrode line.

At least one embodiment of the invention includes an active implantable medical therapy and/or monitoring system that includes at least one implant. In one or more embodiments, the at least one implant includes at least one ultrasonic transducer that may emit and receive ultrasonic signals and that is directly or indirectly connected to a control and evaluation unit. In at least one embodiment, the control and evaluation unit may prompt an emission of ultrasonic signals by the at least one ultrasonic transducer cyclically or in a triggered manner and may evaluate received ultrasonic signals. As such, in at least one embodiment, the control and evaluation unit identifies an actual position or change in position of the implant and/or of another system component of the active implantable medical therapy and/or monitoring system, such as an electrode line.

One or more embodiments of the invention may timely and reliably identify an unintentional change in position, for all electronic implants and electrode lines, via the implant itself.

In at least one embodiment, the active implantable medical therapy and/or monitoring system, for example, may include an active electronic implant that is permanently implanted in the human body. In one or more embodiments, the active electronic implant may be directly or indirectly connected to at least one ultrasonic transducer and to a control and evaluation unit. In at least one embodiment, the control and evaluation unit may be connected to the at least one ultrasonic transducer such that the control and evaluation unit emits and receives ultrasonic signals cyclically or in a triggered manner and evaluates the received signals in order to confirm the implanted position of the implant and/or the electrode line or in order to identify a dislocation.

By way of at least one embodiment, the system may include a device that identifies electrode or implant position errors, wherein at least one ultrasonic transducer is connected to a housing of the implant or to the electrode line. As such, in one or more embodiments, using a measurement of one or more of the ultrasound reflection (amplitudes), signal propagation times and frequency shifts, an inadmissible change in position of the electrode line or the implant is identified.

In at least one embodiment, the device may identify, timely and reliably, unintentional changes in position of an electronic implant or an implanted electrode line.

One or more embodiments of the invention incorporate the finding that typical methods for electrode or implant position error identification are limited in terms of their sensitivity and specificity due to their indirect measurement methods. Furthermore, typical methods may be used only with implantable systems in which, due to design, the parameters listed above may be detected.

According to one or more embodiments, the at least one ultrasonic transducer may include or may be a piezoelectric transducer that may emit and receive ultrasonic signals and that is connected to the control and evaluation unit. In at least one embodiment, the piezoelectric transducer may be used equally in order to emit ultrasonic signals as a transmitter, such as a loudspeaker, and to receive ultrasonic signals as a receiver, such as a microphone. In one or more embodiments, the piezoelectric transducer may be operated efficiently and in a desired sound frequency range.

By way of at least one embodiment, the control and evaluation unit may include a memory or may be connected to a memory. In one or more embodiments, a reference signal may be stored in the memory, and the control and evaluation unit may compare a respective received ultrasonic signal with the reference signal in order to thus identify a change in position of the implant and/or of a further component of the therapy and/or monitoring system. In at least one embodiment, the stored reference signal may be predefined or for example may be recorded at a moment in time at which the implant or a further component of the therapy and/or monitoring system is disposed at a desired location. In one or more embodiments, if a subsequently received ultrasonic signal deviates significantly from the reference signal, the deviation is an indication that the position of the implant and/or the further component of the therapy and/or monitoring system has changed. In at least one embodiment, the implant may include one or more ultrasonic transducers, and the control and evaluation unit may initially record and store an ultrasonic reference for the implantation site, such as an ultrasonic fingerprint, and may then compare the ultrasonic reference with cyclical measurements. According to one or more embodiments, if the actual measurement deviates from the reference pattern beyond a certain measure, the implant may indicate a possible change in position. In at least one embodiment, a rotation of an implant may be identified.

In one or more embodiments of the invention, the therapy and/or monitoring system may include a plurality of ultrasonic transducers that emit and receive ultrasonic signals. In at least one embodiment, the ultrasonic transducers may be connected immovably to the implant or to the further component of the therapy and/or monitoring system. In one or more embodiments, the control and evaluation unit may store, as reference signals, ultrasonic signals received by the ultrasonic transducers at a first moment in time and may compare the reference signals with ultrasonic signals received by the ultrasonic transducers at one or more later moments in time (occurring after the first moment in time). In at least one embodiment, the control and evaluation unit may determine, based on the comparison, whether differences between currently received ultrasonic signals and the reference signals indicate a change in position of the implant and/or of the further component of the therapy and/or monitoring system.

By way of one or more embodiments, the therapy and/or monitoring system may include at least two ultrasonic transducers, wherein the at least two ultrasonic transducers are mounted such that the position may be determined 2-dimensionally or 3-dimensionally based on an ultrasonic echocardiogram or ultrasonic signal (2D or 3D ultrasound hearing).

In at least one embodiment of the invention, the therapy and/or monitoring system may be or may include a heart therapy system wherein, in addition to the implant, may include a further component such as an electrode line which is connected to the implant. In one or more embodiments, the control and evaluation unit may detect a change in position of the electrode line, such as one or more electrode poles of the electrode line. Accordingly, in at least one embodiment, the at least one ultrasonic transducer may be arranged on or in an electrode line, such as close to an electrode pole of the electrode line.

In one or more embodiments, the therapy and/or monitoring system may be a heart therapy system that includes an electrode line and at least two ultrasonic transducers. In at least one embodiment, at least one ultrasonic transducer of the at least two ultrasonic transducers is arranged on or in the implant, such as the cardiac pacemaker. In one or more embodiments, the at least one second ultrasonic transducer of the at least two ultrasonic transducers is arranged on or in the electrode line and both ultrasonic transducers are connected to the control and evaluation unit. As such, in at least one embodiment, one of the at least two ultrasonic transducers may emit ultrasonic signals triggered by the control and evaluation unit, and wherein the at least one other (second) ultrasonic transducer may receive the ultrasonic signals. In one or more embodiments, the control and evaluation unit may detect and may evaluate a signal propagation time between the emission of an ultrasonic signal by one of the ultrasonic transducers and receipt of the ultrasound by the other ultrasonic transducer of the at least two ultrasonic transducers. In at least one embodiment, based on the signal propagation time, optionally with consideration of the phase position of the signal, the control and evaluation unit may easily determine changes in the distance between the implant and the electrode line. In one or more embodiments, the changes may provide an indication of a dislocation of the electrode line. In at least one embodiment, the implantable system may measure the ultrasound propagation time from the electrode to the implant housing or vice versa, and may evaluate the course over time thereof, such that for example the movement of an electrode positioned in the heart may be assessed.

By way of one or more embodiments, at least one ultrasonic transducer may be arranged in or on the electrode line and may be connected to the control and evaluation unit, such that the at least one ultrasonic transducer may emit an ultrasonic signal in response to a signal of the control and evaluation unit and may receive reflected ultrasonic signal components of the emitted ultrasonic signal. In at least one embodiment, the control and evaluation unit may evaluate the reflected ultrasonic signals. As such, in one or more embodiments, with the aid of only a single ultrasonic transducer in the electrode line, the system, such as the control and evaluation unit, may determine a distance between the electrode line and an implant or other structures based on reflected ultrasonic signal components. By way of at least one embodiment, the ultrasonic transducer may be located on the electrode line and may measure the typical reflection of the ultrasonic waves at the implant housing in order to determine position. One or more embodiments of the invention may include two ultrasonic transducers, wherein both ultrasonic transducers may be arranged in the electrode line, and wherein one of ultrasonic transducers emits ultrasonic signals, and the other of the two ultrasonic transducers receives the reflected signal components.

In at least one embodiment, the control and evaluation unit may evaluate received ultrasonic signals in terms of one or more of propagation time, frequency shift, amplitude and phase position.

In one or more embodiments, the control and evaluation unit may detect frequency shifts between the frequency of received ultrasonic signals and the frequency of emitted ultrasonic signals in order to determine the presence or absence of a Doppler effect, for example to determine whether an electrode line is located in a flowing medium, such as blood. In at least one embodiment, the control and evaluation unit may evaluate the Doppler effect in order to thus determine whether the implant is still fixed to a moving target tissue or if the implant is located in or at a bloodstream path. In one or more embodiments, the control and evaluation unit may evaluate the Doppler effect such that the control and evaluation unit determines whether or not the implant is located in a bloodstream path.

In at least one embodiment, the therapy and/or monitoring system may include a heart stimulator and/or a heart monitor as the implant, wherein the control and evaluation unit may evaluate received ultrasonic signals under consideration of physiological signals received from the implant and representing a respective heart cycle. In one or more embodiments, the physiological signals may be, for example, intracardial electrocardiograms, which represent the heart cycle. As such, in at least one embodiment, the system, such as the control and evaluation unit, may correlate movements within a heart chamber, which also lead to cyclical changes in position of the respective electrode line, with the electrophysiological signals of the heart. In one or more embodiments, the system, such as the control and evaluation unit, may determine that the changes in position measured by ultrasound correspond to the expected changes in position as a result of the movement of the heart, such that such a determination is an indication of an electrode line that has not been dislocated.

According to one or more embodiments, the control and evaluation unit may generate a movement profile of the implant from received ultrasonic signals and may evaluate the movement profile in relation to electrophysiological signals representing a heart cycle. In at least one embodiment, the ultrasonic evaluation of an implant located at or in the bloodstream path may be supplemented by the evaluation of the heart cycle, such as using an ECG unit, in order to evaluate the movement profile of the blood flow or of the implant with regard to possible dislocations (for example the dislocation of a right-ventricular leadless pacemaker in the pulmonary artery or the atrium).

### BRIEF DESCRIPTION OF THE DRAWINGS

The above and other aspects, features and advantages of at least one embodiment of the invention will be more apparent from the following more particular description thereof, presented in conjunction with the following drawings, wherein:

FIG. 1 shows a schematic block diagram of an implant according to embodiments of the invention;

FIG. 2 shows a first application example of a dislocation sensor for an ultrasonic Doppler measurement in the blood vessel;

FIG. 3 shows a further implementation example for a propagation time measurement;

FIG. 4 shows a further embodiment of the dislocation identification for ultrasound stereo hearing; and

FIGS. 5A and 5B show a single dislocation sensor with ultrasonic reference; according to one or more embodiments of the invention.

### DETAILED DESCRIPTION OF THE INVENTION

The following description is of the best mode presently contemplated for carrying out at least one embodiment of the invention. This description is not to be taken in a limiting sense, but is made merely for the purpose of describing the general principles of the invention. The scope of the invention should be determined with reference to the claims.

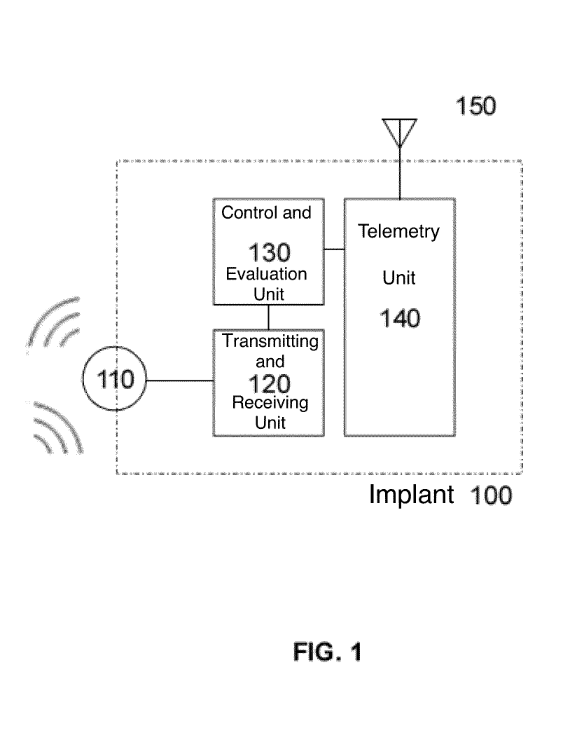

FIG. 1 shows an active implantable medical therapy and/or monitoring system as an implant 100, according to one or more embodiments of the invention. FIG. 1 shows the block diagram of the implant 100, according to at least one embodiment of the invention. As shown in FIG. 1, in one or more embodiments, the implant 100 includes one or more ultrasonic transducers 110, mounted on the housing of the implant 100 or as a component of an electrode line connected to the implant 100. In at least one embodiment, the ultrasonic transducer 110 is connected to an ultrasound transmitting and receiving unit 120, which is in turn connected to a control and evaluation unit 130. In one or more embodiments, the control and evaluation unit 130 may start an ultrasound scan, either cyclically or triggered by certain events, and may evaluate the received ultrasonic signals in order to assess the current position of the implant or of the electrode line. In at least one embodiment, the assessment may be performed based on one or more of ultrasound propagation times, frequency shifts (Doppler effect), amplitude values and phase positions. One or more embodiments may include a telemetry unit 140 with antenna 150, such as part of the implant 100, wherein the telemetry unit may be connected to the control and evaluation unit 130, such that an identified dislocation may be signaled accordingly.

In at least one embodiment, the implant may include one or more units used to provide therapy or monitoring.

In one or more embodiments, the ultrasonic frequencies to be used may lie in the region of ˜1 MHz, such that a penetration depth of approximately 50 cm is provided. In at least one embodiment, the ultrasonic frequencies used may depend on application-specific differences.

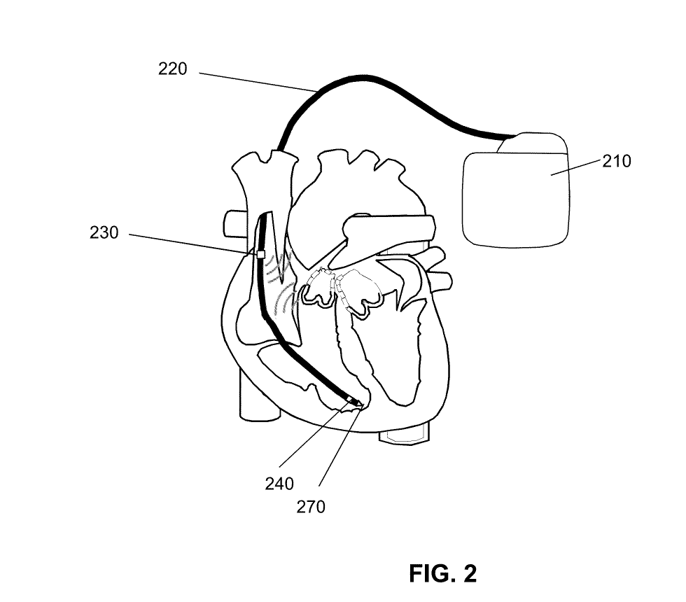

FIG. 2 illustrates a first application example of a dislocation sensor, according to one or more embodiments of the invention. At least one embodiment may include an implantable cardiac pacemaker 210 connected to an implantable electrode line 220, wherein the implantable electrode line 220 may include an ultrasonic transducer 230. In one or more embodiments, a stimulation electrode pole that includes a ring electrode 240 and a tip electrode 270 may be located in the distal region of the electrode.

In at least one embodiment, the ultrasonic transducer 230, together with the associated ultrasound transmitting or receiving unit and the control and evaluation unit, may establish the blood flow in the vena cava using a heart frequency-synchronous measurement and frequency evaluation (Doppler effect), such that the position of the electrode is confirmed.

In one or more embodiments, the heart frequency-synchronous measurement may be applied to identify the dislocation of pulmonary artery pressure sensors.

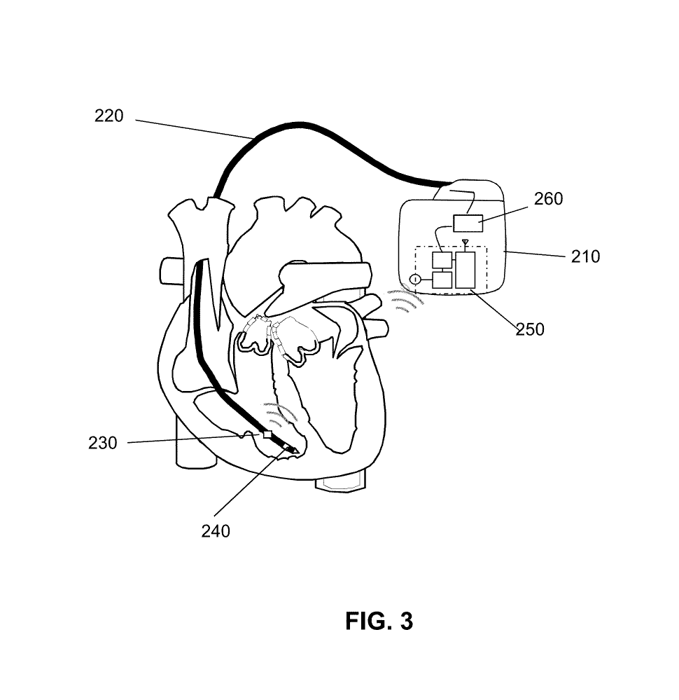

FIG. 3 shows a further implementation example for a propagation time measurement, according to one or more embodiments of the invention. In at least one embodiment, the implant 210 may be connected to an electrode line 220 that includes a mini piezo ultrasound transmitter 230 and a stimulation electrode pole 240. In one or more embodiments, the ultrasound transmitter 230 may be mounted in the vicinity of the stimulation electrode pole 240. In at least one embodiment, an ultrasound receiver 250 may be mounted on the implant housing 210.

By way of one or more embodiments, in order to assess the position of the stimulation electrode pole 240, the ultrasound propagation time between transmitter 240 and receiver 250 may be evaluated. In at least one embodiment, if the ultrasound propagation time between transmitter 240 and receiver 250 changes beyond an admissible measure, a dislocation of the electrode tip is indicated.

In order to improve the specificity and sensitivity, in one or more embodiments, the propagation time measurement may be performed continuously over a heart cycle. In at least one embodiment, a differentiation of a micro-dislocation with additional secondary electrode movements, such as unphysiological “wobbling” of the electrode tip, may be performed based on a movement curve obtained therefrom.

In one or more embodiments, the implant 210 may include a detection unit 260 that records an intracardial electrocardiogram, which evaluates an electrophysiological signal recorded via the electrode pole 240 or 270.

In at least one embodiment, the receiver 250, for example, may correspond to the ultrasonic transducer 110 and the ultrasound transmitting and receiving unit 120 from FIG. 1. In one or more embodiments, the control and evaluation unit 130 may be connected to the detection unit 260.

In at least one embodiment, the transmitting ultrasonic transducer and the receiving ultrasonic transducer may be swapped, wherein the transmitter is the ultrasonic transducer 250 and the receiver is the ultrasonic transducer 230.

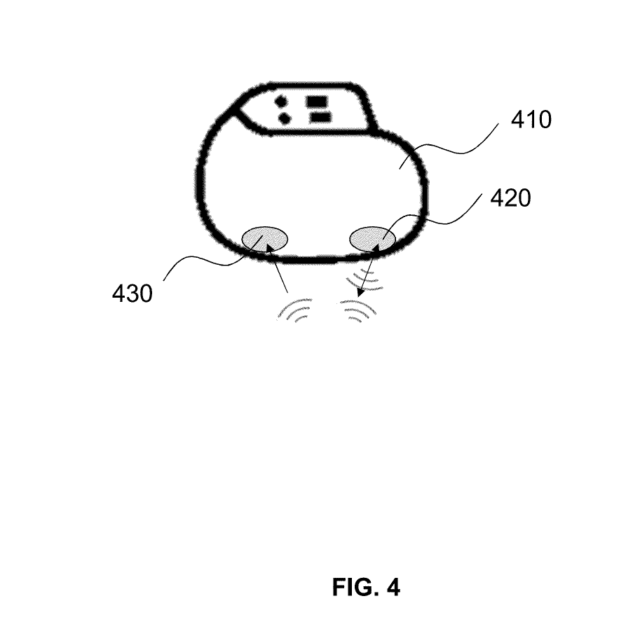

FIG. 4 illustrates a further embodiment of the dislocation identification for ultrasound stereo hearing, according to one or more embodiments of the invention. In at least one embodiment, as shown in FIG. 4, at least two piezo ultrasonic transducers 420 and 430 together with associated ultrasound transmitting or receiving unit may be mounted on the housing of an implant 410. In one or more embodiments, the first ultrasonic transducer 420 may be used to emit an ultrasonic pulse, wherein the reflections of the ultrasonic pulse may be received by the first ultrasonic transducer 420 and the second ultrasonic transducer 430. In at least one embodiment, a 2-dimensional classification of the ultrasound reflection may be made based on a comparison of the two receivers. For example, in one or more embodiments, the echo of a reflection surface on an electrode may be measured.

One or more embodiments may include further receivers, for example for 3-dimensional classification. In at least one embodiment, the ultrasonic transducers may be provided in the electrode line, and the housing 410 may serve as a reflector for the ultrasound. In one or more embodiments, the ultrasonic transducers may be positioned sufficiently far from one another. By way of at least one embodiment, a very large characteristic echo from the implant housing 410 may be recorded. According to one or more embodiments, the position of a number of electrode portions may be checked at the same time.

At least one embodiment of the invention may include a separate echo reflector (not illustrated in the Figures) implanted as a position reference.

One or more embodiments of the invention may include a combination of elements of FIG. 4 and elements of FIG. 3. In at least one embodiment, the electrode line may include at least one mini piezo ultrasound transmitter (such as element 230 of FIG. 3), and at least two piezo ultrasound receivers (such as elements 420, 430 of FIG. 4) mounted on the implant. As such, in one or more embodiments, an improvement of the specificity and sensitivity of the dislocation identification may be made possible by evaluation of propagation time and amplitude differences as well as with use of the Doppler effect as a result of the moving electrode. In at least one embodiment, the ultrasound transmitter 230 and ultrasound receivers 420 and 430 each have an ultrasonic transducer such as the ultrasonic transducer 110 from FIG. 1 as well as an ultrasound transmitting or receiving unit such as the ultrasound transmitting and receiving unit 120 from FIG. 1.

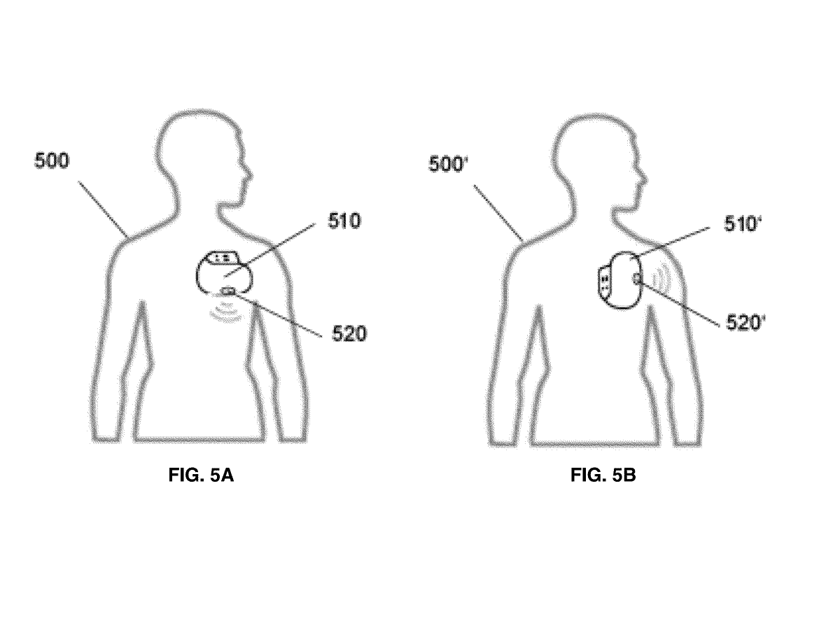

FIGS. 5A and 5B show a dislocation sensor with ultrasonic reference, according to one or more embodiments of the invention. As shown in FIGS. 5A and 5B, in at least one embodiment, the dislocation sensor may include or may be only one single ultrasonic transducer 520, 520′ on the implant 510, 510′. In at least one embodiment, the ultrasonic transducer 520, 520′ may be part of a sensor that includes an ultrasound transmitting and receiving unit 120 and a control and evaluation unit 130, which records an ultrasound reflection reference with a determined implant position in the patient 500, shown in FIG. 5A, and stores the ultrasound reflection reference. In one or more embodiments, for the subsequent implant monitoring, additional ultrasonic measurements may be taken cyclically and may be compared with the recorded ultrasound reflection reference. In at least one embodiment, if the deviations from the ultrasound reflection reference exceed a limit value, a dislocation may assumed and signaled, as shown in FIG. 5B. In one or more embodiments, the constructions shown in FIGS. 5A and 5B may be used when a twisting of an implant (for example a heart monitor) is to be identified.

By way of at least one embodiment, active implants with the described ultrasonic dislocation sensor may be insulin pumps, ventricular assist devices (VADs), implantable monitors, PillCam implants, neurostimulators, stimulators that provide cardial resynchronization, implantable controllers for orthopedic implants, and retina implants.

In one or more embodiments, the ultrasonic monitoring of the implant may provide an active monitoring of only passive implants, such as orthopedic prostheses.

In at least one embodiment, ultrasonic transducers used in medical ultrasonic diagnosis systems, such as 3D and 4D echocardiography with matrix technology, may be miniaturized to a size of, for example, 350 μm and may be used without limitation for all specified implant applications described herein.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

### LIST OF REFERENCE SIGNS

100 implant
110 ultrasonic transducer
120 ultrasound transmitting and receiving unit
130 control and evaluation unit
140 telemetry unit
150 antenna
210 cardiac pacemaker
220 electrode line
230 ultrasonic transducer
240 stimulation electrode pole
250 ultrasound receiver
260 electrocardiogram detection unit
270 tip electrode pole
410 implant
420 ultrasonic transducer
430 ultrasonic transducer
500 patient
510 implant
520 ultrasonic transducer

What is claimed is:

1. An active implantable medical therapy and/or monitoring system comprising:
at least one implant, wherein the at least one implant comprises
at least one ultrasonic transducer that emits and records ultrasonic signals,
a control and evaluation unit directly or indirectly connected to the at least one ultrasonic transducer,
wherein the control and evaluation unit
prompts an emission of ultrasonic signals by the at least one ultrasonic transducer cyclically or in a triggered manner,
evaluates received ultrasonic signals, and,
identifies an actual position or change in position of one or more of the at least one implant and a further component of the active implantable medical therapy and/or monitoring system.

2. The active implantable medical therapy and/or monitoring system according to claim 1, wherein the at least one ultrasonic transducer is a piezoelectric transducer that emits and receives ultrasonic signals, and wherein the piezoelectric transducer is connected to the control and evaluation unit.

3. The active implantable medical therapy and/or monitoring system according to claim 1, wherein the control and evaluation unit comprises or is connected to a memory, wherein the memory stores a reference signal, wherein the control and evaluation unit compares a respective received ultrasonic signal with the reference signal and identifies a change in position of one or more of the at least one implant and the further component of the active implantable medical therapy and/or monitoring system.

4. The active implantable medical therapy and/or monitoring system according to claim 1, wherein the active implantable medical therapy and/or monitoring system is a heart therapy system and further comprises an electrode line.

5. The active implantable medical therapy and/or monitoring system according to claim 4, wherein the electrode line comprises an electrode pole, wherein the at least one ultrasonic transducer is arranged on or in the electrode line after the electrode pole.

6. The active implantable medical therapy and/or monitoring system according to claim 4, further comprising at least one second ultrasonic transducer arranged in or on the at least one implant,
wherein the at least one ultrasonic transducer is connected on or in the electrode line to the control and evaluation unit, and
wherein the at least one ultrasonic transducer, triggered by said control and evaluation unit, emits ultrasonic signals, and the at least one second ultrasonic transducer in or on the at least one implant receives ultrasonic signals, or
wherein the at least one ultrasonic transducer, triggered by said control and evaluation unit, receives ultrasonic signals, and the at least one second ultrasonic transducer in or on the at least one implant emits ultrasonic signals, and
wherein the control and evaluation unit detects and evaluates a signal propagation time between an emission of an ultrasonic signal by the at least one ultrasonic transducer or the at least one second ultrasonic transducer and receipt of the ultrasonic signal by another of the at least one ultrasonic transducer or the at least one second ultrasonic transducer. The active implantable medical therapy and/or monitoring system according to claim 5, wherein the at least one ultrasonic transducer arranged in or on the electrode line is connected to the control and evaluation unit, wherein the at least one ultrasonic transducer emits an ultrasonic signal in response to a signal of the control and evaluation unit and receives reflected ultrasonic signal components of the emitted ultrasonic signal, and wherein the control and evaluation unit evaluates the reflected ultrasonic signal components.

8. The active implantable medical therapy and/or monitoring system according to claim 1, wherein the at least one ultrasonic transducer comprises a plurality of ultrasonic transducers that emit and receive ultrasonic signals,
wherein the plurality ultrasonic transducers are connected immovably to the at least one implant,
wherein the control and evaluation unit
stores, as reference signals, ultrasonic signals received by the plurality of ultrasonic transducers at a first moment in time,
compares the reference signals with ultrasonic signals received by the plurality of ultrasonic transducers at one or more later moments in time, and
determines, based on the comparison, whether differences between currently received ultrasonic signals and the reference signals indicate a change in position of the at least one implant.

9. The active implantable medical therapy and/or monitoring system according to claim 1, wherein the control and evaluation unit evaluates received ultrasonic signals in terms of one or more of propagation time, frequency shift, amplitude and phase position.

10. The active implantable medical therapy and/or monitoring system according to claim 1, wherein the control and evaluation unit detects frequency shifts of received ultrasonic signals compared to frequency of emitted ultrasonic signals to determine a presence or absence of a Doppler effect.

11. The active implantable medical therapy and/or monitoring system according to claim 1, wherein the at least one implant is one or more of a heart stimulator and a heart monitor, and wherein the control and evaluation unit evaluates received ultrasonic signals under consideration of physiological signals received by the at least one implant and that represent a respective heart cycle.

12. The active implantable medical therapy and/or monitoring system according to claim 11, wherein the control and evaluation unit generates a movement profile of the at least one implant from received ultrasonic signals and evaluates the movement profile in relation to electrophysiological signals representing a heart cycle.
